(19) United States
(12) Patent Application Publication
(10) Pub. No.: US 2011/0104313 A1
(43) Pub. Date: May 5, 2011

(54) COMPOSITIONS FOR THE TREATMENT OF VAGINAL INFECTIONS WITH CHRONIC INFLAMMATION

(75) Inventors: Ezio Bombardelli, Gropello Cairoli (IT); Gabriele Fontana, Milano (IT); Andrea Giori, Milano (IT); Paolo Morazzoni, Milano (IT); Antonella Riva, Milano (IT); Massimo Ronchi, Milano (IT)

(73) Assignee: INDENA S.P.A, Milano (IT)

(21) Appl. No.: 12/988,897

(22) PCT Filed: Apr. 6, 2009
(86) PCT No.: PCT/EP09/02516

§ 371 Date: Dec. 16, 2010

(30) Foreign Application Priority Data
Apr. 24, 2008 (IT) MI2008A000751
Jun. 12, 2008 (EP) 08425421.8

(51) Int. Cl. A61K 36/28; A61K 36/758; A61K 36/66; A61P 29/00; A61P 31/00

(52) U.S. Cl. 424/737; 424/749

(57) ABSTRACT

The present invention relates to compositions comprising benzofuran compounds and benzophenanthridine alkaloids, which possess anti-inflammatory, antibacterial and antifungal activity useful in the treatment of vaginal infections and the resulting inflammatory states.

### SUMMARY

The present invention relates to compositions comprising benzofuran compounds and benzophenanthridine alkaloids, which possess anti-inflammatory, antibacterial and antifungal activity useful in the treatment of vaginal infections and the resulting inflammatory states.

### PRIOR ART

Vaginitis is often initially asymptomatic, but with time can degenerate into infections which may be dangerous. Vulvovaginal infections, whether they are of viral, bacterial, fungal or protozoal origin (Herpes, trichomoniasis, candidiasis) cause vulvar itching, stinging, irritation and lesions, followed by external dysuria and vulvar dyspareunia. Vaginitis can lead to a series of serious events, with recurrent infections, such as toxicity to other organs and apparatus. This phenomenon is particularly important in many developing countries, where these events predispose the sufferer to the risk of contracting HIV or other sexually transmissible diseases.

Trichomoniasis presents symptoms such as a yellowish, purulent exudate with vulvar irritation, inflammation of the vaginal and vulvar epithelium, and petechial lesions of the cervix. The pH of the secretion is greater than 5, thus promoting the development of Trichomonas. In candidiasis there is severe vulvar itching with erythema and oedema, and the secretions are foul-smelling, as in the case of bacterial vaginitis. These disorders are treated with oral antibiotics and antifungals administered at high doses, or with gels for local treatment. These treatments always take a long time, and can have side effects.

The benzophenanthridine alkaloids isolated from Macleaya cordata, Macleaya microcarpa, Sanguinaria canadensis and Chelidonia majus are particularly active on strains directly involved in vaginal infections, such as Trichomonas vaginalis, Escherichia coli, Pseudomonas aeruginosa and the like.

According to the invention, the benzofuran compounds have the following formula

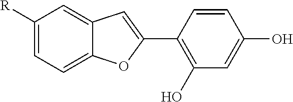

where R may be hydrogen or a linear or branched alkyl chain with 2 to 6 carbon atoms, or an alkyl chain substituted by amine, nitro groups; R is preferably hydrogen or alkyl C1-C3.

Said benzofuran compounds are known and can be prepared by conventional methods, for example by reaction of a phenol suitably substituted with 2-phenoxy-2′,4′-dimethoxyacetophenone in the conditions reported in Chimie Therapeutique 1973, 8, 398, followed by cyclisation in the presence of polyphosphoric acid in xylene and hydrolysis of the methoxy and hydroxy groups. The benzofuran compounds used in the compositions according to the invention have structural formula 1 and possess a powerful antibacterial and antifungal action against numerous strains of Candida.

### DESCRIPTION OF THE INVENTION

The present invention relates to compositions comprising:
a) benzophenanthridine alkaloids; and
b) benzofuran compounds;
and possibly
c) extract of Zanthoxylum bungeanum or Echinacea angustifolia;

which possess anti-inflammatory, antibacterial and antifungal activity useful in the treatment of vaginal infections and the resulting inflammatory states, especially vaginitis of various origins with associated inflammatory problems.

More particularly, the present invention relates to formulations comprising:
a) benzophenanthridine alkaloids selected from sanguinarine and/or chelerythrine and/or derivatives thereof; and
b) benzofuran compounds as specified above;
and possibly
c) extract of Zanthoxylum bungeanum or Echinacea angustifolia.

It has now surprisingly been found that the compositions according to the invention possess an antibacterial, antifungal and antienzymatic activity greater than that of the sum of the various components administered separately. Said effect may be due to a synergistic action mechanism which takes place between the various components of the association in question. The compositions according to the invention rapidly eliminate these infections, eliminating the presence of the saprophyte and reducing inflammation, itching and the vaginal pH.

According to the invention, the compositions will contain the various components in the following intervals (by weight per unit dose):
a) benzophenanthridine alkaloids: from 0.15 mg to 15 mg; and
b) benzofuran compounds: from 0.2 to 25 mg;
and possibly
c) extract of Zanthoxylum bungeanum or Echinacea angustifolia: from 0.1 to 10 mg.

According to a particularly preferred aspect, the compositions in question will contain the various components within the following intervals (by weight per unit dose):
a) benzophenanthridine alkaloids: from 0.4 to 10 mg; and
b) benzofuran compounds: from 0.4 to 10 mg;
and possibly
c) extract of Zanthoxylum bungeanum or Echinacea angustifolia: from 0.2 to 5 mg.

The benzophenanthridine alkaloids sanguinarine and chelerythrine may be present in free or salified form, as such in substantially pure form or in the form of extracts of Sanguinaria canadensis, Macleaya cordata, Macleaya microcarpa or Chelidonia majus. According to a preferred aspect, the benzophenanthridine alkaloids will be present in a form salified with luteic acid. Said salts, which are prepared by reacting the sulphates or chlorides of the alkaloids with the sodium or potassium salt of luteic acid and subsequent crystallisation, have proved particularly effective for the purposes of this invention. In particular, sanguinarine is a powerful anti-angiogenesis factor which helps to reduce inflammation (Jong-Pil Eun 2004). In vivo, sanguinarine suppresses capillary formation in Matrigel and in the chorioallantoic membrane in chicken embryo. (Jong-Pil Eun 2004).

Said benzophenanthridine alkaloids not only have considerable antibacterial, antifungal, and antitrichomonas activity, but also present considerable activity against cytomegalovirus and papillomavirus. For this reason the archetypes of these alkaloids, sanguinarine, chelerythrine and chelidonine, which also have an analgesic effect, are very useful in the treatment of vaginitis of different etiologies. These compounds act in synergy with one another to reduce inflammation, and consequently the symptoms, and to suppress the disorder.

The compounds with a benzofuran structure described above may be present as such or in the form of extracts containing them, such as extracts of Krameria triandra, Eupomatia laurina and Piper sp. The compounds isolated from Krameria triandra which have proved particularly active are Eupomatenoid 6 and neolignan 2-(2,4-dihydroxyphenyl)-5-(E)-propenyl-benzofuran, which have demonstrated antibacterial and antifungal activity on numerous strains of Gram+ bacteria, fungi and anaerobic bacteria.

According to a particularly preferred aspect, the compositions in question will also contain an extract of Zanthoxylum bungeanum or Echinacea angustifolia to help eliminate itching and/or pain, when present. This action is due to the presence of isobutylamides which bind the cannabinoid CB2 and CB1 receptors. The formulations according to the invention can be prepared according to well-known conventional methods, such as those described in “Remington's Pharmaceutical Handbook”, Mack Publishing Co., N.Y., USA, together with suitable excipients.

The compositions according to the invention will be conveniently formulated in water/oil emulsions with other compatible excipients for external treatment of the anogenital region; for internal treatments the compounds will be suspended in oils in soft gelatin capsules which disintegrate easily after introduction into the vaginal meatus.

Examples of formulations according to the invention include creams, ointments, powders, lotions and the like, vaginal pessaries or equivalent formulations, including capsules that dissolve at internal body temperature.

The examples set out below illustrate the invention, without limiting its scope.

### Example 1

### Preparation of Benzofuran Compounds

### Stage A. Preparation of 2-phenoxy-2′,4′-dimethoxyacetophenone (a)

A solution of 2-bromo-2′,4′-dimethoxyacetophenone (5 g, 19.1 mmols) in 25 mL of 2-butanone was added to a suspension of phenol (1.8 g, 19.1 mmols), K₂CO₃ (2.6 g, 19.1 mmols) and KI (41.5 mg, 0.25 mmols) in 20.0 mL of the same solvent. The solution was then refluxed for 20 hours. The mixture was filtered and the solvent was eliminated under vacuum. The residue obtained was dissolved in EtOAc and washed with a 10% aqueous solution of NaOH and then with water. The organic extract was dried over Na₂SO₄, filtered and evaporated under vacuum. Finally, the crude residue was washed with Et₂O and dried under low pressure to provide 4.4 g (yield: 84%) of the title compound.

### Stage B. Preparation of 2-(2′,4′-dimethoxyphenyl)benzofuran (b)

12 g of polyphosphoric acid was added to a solution of the compound obtained at Stage A (4.4 g, 16.2 mmols) in 130.0 mL of xylene. The mixture was refluxed for 2 hours, and then left to cool at ambient temperature. The solution was then decanted and evaporated under low pressure. The resulting residue (3.7 g, yield: 90%) was used at the next stage without further purification.

### Stage C. Preparation of 2-(2′,4′-dihydroxyphenyl)benzofuran (1)

A mixture of the compound prepared at Stage B (3.7 g, 14.5 mmols) and pyridine hydrochloride (11.1 g, 96.4 mmols) was heated to 225° C. for 45 minutes. The red product formed was poured into 10% HCl. The mixture was washed repeatedly with EtOAc; the combined organic layers were dried over Na₂SO₄ and evaporated. The residue was purified by column chromatography (hexane/EtOAc=7:3) to provide. The final compound was obtained with a yield of 41% (1.36 g) after crystallisation from benzene.

### Formulation Example 1

### Oily Suspension for Soft Gelatin Capsules to be Inserted in the Vaginal Meatus

| | |
|---|---|
| Macleaya cordata lipophilic extract (75%) | 10 mg |
| 2,4-Dihydroxyphenyl-3-benzofuran | 10 mg |
| Soya lecithin | 60 mg |
| Beeswax | 50 mg |
| Vegetable oil q.s. to | 800 mg |

### Formulation Example 2

### Cream (Oil-in-Water Emulsion)

| | |
|---|---|
| Extract of Krameria triandra | 0.4 g |
| Macleaya cordata alkaloid fraction | 0.4 g |
| Zanthoxylum bungeanum lipophilic extract | 0.2 g |
| Propylene glycol | 10.00 g |
| Isopropyl myristate | 5.00 g |
| Cetyl alcohol | 5.00 g |
| Polysorbate 80 | 3.00 g |
| Carbomer | 0.40 g |
| Methyl parahydroxy benzoate | 0.10 g |
| Propyl parahydroxy benzoate | 0.05 g |
| Purified water q.s. to | 100 g |

### Formulation Example 3

### Vaginal Pessary

| | | |
|---|---|---|
| 2,4-Dihydroxyphenyl-3-benzofuran | 10 | mg |
| Macleaya alkaloid fraction | 3 | mg |
| Glycerides of fatty acids q.s. to | 2.0 | g |

1-11. (canceled)

12. Compositions comprising:
a) benzophenanthridine alkaloids; and
b) benzofuran compounds of formula 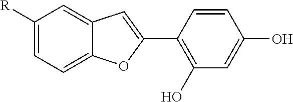
where R may be hydrogen or a linear or branched alkyl chain with 2 to 6 carbon atoms, or an alkyl chain substituted by amine, nitro groups; and
c) extract of Zanthoxylum bungeanum or Echinacea angustifolia.

13. Compositions as claimed in claim 12, wherein the benzophenanthridine alkaloids are selected from sanguinarine, chelerythrine or chelidonine or derivatives thereof.

14. Compositions as claimed in claim 12, wherein by weight per unit dose:
a) benzophenanthridine alkaloids are present from 0.15 mg to 15 mg; and
b) benzofuran compounds are present from 0.2 to 25 mg; and
c) extract of Zanthoxylum bungeanum or Echinacea angustifolia are present from 0.1 to 10 mg.

15. Compositions as claimed in claim 14, wherein by weight per unit dose
a) benzophenanthridine alkaloids are present from 0.4 to 10 mg; and/or
b) benzofuran compounds are present from 0.4 to 10 mg; and
c) extract of Zanthoxylum bungeanum or Echinacea angustifolia are present from 0.2 to 5 mg.

16. Compositions as claimed in claim 12, wherein the benzophenanthridine alkaloids sanguinarine and chelerythrine are in free or salified form, pure form or as extracts of Sanguinaria canadensis, Macleaya cordata, Macleaya microcarpa or Chelidonia majus.

17. Compositions as claimed in claim 16, wherein the benzophenanthridine alkaloids are salified with luteic acid.

18. Compositions as claimed in claim 12, wherein the benzofuran compounds of formula 1 are benzofuran-containing extracts.

19. Compositions as claimed in claim 18, wherein the benzofuran-containing extracts are extracts of Krameria triandra, Eupomatia laurina and Piper sp.

20. Formulations as claimed in claim 12, in the form of water/oil emulsions, soft gelatin capsules, vaginal pessaries or equivalent formulations, creams, ointments, powders, lotions.

21. A method of treatment of vaginal infections and resulting inflammatory states thereof, said method comprising preparing topical formulations comprising
a) benzophenanthridine alkaloids;
b) benzofuran compounds; and
c) extract of Zanthoxylum bungeanum or Echinacea angustifolia;
administering said topical formulations and treating vaginal infections and resulting inflammatory states.

22. The method as claimed in claim 21, wherein the vaginal infections are vaginitis with associated inflammatory problems.
